(19) United States
(12) Patent Application Publication
(10) Pub. No.: US 2005/0175570 A1
(43) Pub. Date: Aug. 11, 2005

(54) Composition containing a semi-crystalline polymer and a polyvinylpyrrolidone/alpha-olefin copolymer

(75) Inventors: Mika Inoue, Tokyo (JP); Noriko Kawashima, Yokohama-shi (JP); Mariko Okamoto, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/014,823

(22) Filed: Dec. 20, 2004

Related U.S. Application Data
(60) Provisional application No. 60/537,559, filed on Jan. 21, 2004.

(30) Foreign Application Priority Data
Jan. 5, 2004 (JP) 2004-000712

(51) Int. Cl. A61K 7/42; A61K 7/06; A61K 7/11

(52) U.S. Cl. 424/70.15

(57) ABSTRACT

A preferred invention composition contains: (A) at least one semi-crystalline polymer having a melting point of at least 50° C., preferably 50-120° C., more preferably 50-70° C.; (B) at least one polyvinylpyrrolidone/α-olefin copolymer having a melting point of at least 50° C., preferably 50-120° C., more preferably 50-70° C.; and (C) an oil phase containing at least one non-volatile oil, and exists in the form of a paste or cream at 25° C.

### REFERENCE TO PRIOR APPLICATIONS

This application claims priority to U.S. provisional application 60/537,559 filed Jan. 21, 2004, and to Japanese patent application 2004-000712 filed Jan. 5, 2004, both incorporated herein by reference.

### FIELD OF THE INVENTION

The present invention relates in general to compositions preferably useful for external application such as cosmetics and the like, and more preferably relates to paste or cream type compositions for external application excelling in usability, having high stability and good applicability to skin and the like. Additionally, the present invention also relates to a method for producing such a composition for external application. A preferred invention composition comprises:

(A) at least one semi-crystalline polymer having a melting point of at least 50° C., preferably 50-120° C., more preferably 50-70° C.;

(B) at least one polyvinylpyrrolidone/α-olefin copolymer having a melting point of at least 50° C., preferably 50-120° C., more preferably 50-70° C.; and

(C) an oil phase containing at least one non-volatile oil.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Oil-based cosmetic compositions can be largely divided into oil-based liquid cosmetic compositions and oil-based solid cosmetic compositions.

For example, while the makeup removing products which are currently marketed generally include those in cream, emulsion, oil and gel form, oil-type makeup removers (so-called cleansing oils) are greatly favored by women for having an extremely high makeup removal effect, being easily massaged into the skin, and not leaving behind oils on the skin after rinsing with water due to the inclusion of surfactants.

Additionally, water-in-oil or non-aqueous foundations in cream, emulsion and liquid form are generally known. Liquid foundations include those in two-layer form, which must be shaken to homogenize immediately before use. Liquid or two-layer foundations are also very much favored for being easy to spread, forming a thin cosmetic film, and having a light sensation of use. Furthermore, sunblocking emulsions in the form of water-in-oil type two layer liquids are also widely used, and are used by shaking to homogenize immediately before use.

However, since these liquid and semi-liquid type products have high fluidity, they can splatter when taken onto the hands, or drip when applied from the hands to the face, thereby soiling clothes. Additionally, such water-in-oil type cosmetic compositions and non-aqueous oil-based products, once stuck to clothes, are much more difficult to remove than water-based cosmetics.

On the other hand, solid cosmetic compositions are common in skin-care products and makeup products, and often come in the form of a stick or a compact. These types of cosmetics are convenient to carry and port, with no risk of spillage.

Most of these solid cosmetic compositions are non-aqueous gels that do not contain water, and such gels, especially when formed into sticks, have the drawback of being difficult to spread when applied to the skin due to their property of hardness. Additionally, when formed into a cast compact type, a sponge is needed for application to the skin. Furthermore, with these products, the stick and skin or sponge and skin come into direct contact during application to the skin, which is not desirable in terms of sanitation.

For further background, note JP-A 2003-40739 and JP-A H7-89826.

The present inventors attempted to overcome the drawbacks possessed by the oil-based liquid cosmetic compositions by constructing an oil phase in an oil-based cosmetic composition using a thickener or the like, but were not able to obtain satisfactory results.

### OBJECTS OF THE INVENTION

Consequently, a main object of the present invention is to provide a composition for external application such as a non-solid cosmetic composition which overcomes the problems in the above-described conventional art, and a method for production thereof.

Another object of the present invention is to provide a composition for external application in the form of a paste or a cream excelling in stability, particularly in stability at high temperatures, and a method for production thereof.

A further object of the present invention is to provide a composition for external application among oil-based cosmetic compositions containing an emulsion system, providing effects such as enabling a suitable amount to be readily taken onto a finger, being readily spread when applied to the skin, and being light to the touch, due to being in paste or cream form, and a method for production thereof.

### DETAILED DESCRIPTION OF THE PREFERRED EMBODIMENTS

The present inventors discovered, to their surprise, that by using two special types of polymers to gelify a non-volatile oil phase, and destroying the gel structure thereof, or suppressing the progress of gelification, it is possible to obtain a cosmetic composition excelling in stability, and also having at least some of the various desirable properties described above.

Thus, the present invention prvides a composition, preferably suitable for external application, comprising:

(A) at least one semi-crystalline polymer having a melting point of at least 50° C., preferably 50-120° C., more preferably 50-70° C.;

(B) at least one polyvinylpyrrolidone/α-olefin copolymer having a melting point of at least 50° C., preferably 50-120° C., more preferably 50-70° C.; and

(C) an oil phase containing at least one non-volatile oil.

In a highly preferred embodiment the compositon is one taking a paste or cream form at room temperature (25° C.).

The composition according to the present invention has an optimum viscosity to excel in usability, has high stability, especially high-temperature stability, is especially easy to take a suitable amount onto a finger, spreads well when applied to the skin, and/or is readily massaged onto the skin, and has a light touch.

The fact that liquid oil phases can be structuralized by using semi-crystalline polymers to obtain cosmetic compositions such as stick-type lip rouges is already known. For example, JP-A 2003-40739 discloses a composition essentially comprising a semi-crystalline polymer with a low melting point of less than 50° C. and a optionally comprising a semi-crystalline polymer having a high melting point of at least 50° C. However, in order to obtain a paste or cream type composition, simply reducing the amount of the semi-crystalline polymer die not result in stability, especially high-temperature stability at 45° C. or more.

Additionally, the use of a vinylpyrrolidone copolymer to structuralize a liquid oil phase to obtain a stick-type cosmetic composition is also known (e.g. see JP-A H7-89826). Therefore, attempts have been made to obtain a paste or cream type product by simply reducing the amount of vinylpyrrolidone copolymer, but in this case also, the product had poor stability, especially high-temperature stability at 45° C. or more.

Thhe composition of the present invention can be produced by a method comprising blending at least one semi-crystalline polymer having a melting point of at least 50° C., and at least one polyvinylpyrrolidone/α-olefin copolymer having a melting point of at least 50° C., with an oil phase comprising at least one non-volatile oil; gelifying said oil phase; and applying a shear force after gelification or during gelification to obtain a fragmented gel structure.

In the present invention, the gel structure may be fragmented by any method, and it may take many forms depending on the production capacity, manner of use of the subject composition, the types and amounts of optional ingredients contained in the composition, and types of apparatus which can be used.

For example, in one embodiment, the composition according to the present invention is formed by blending a semi-crystalline polymer and a polyvinylpyrrolidone/α-olefin copolymer with an oil phase, then gelifying the oil phase to prepare a solid gel composition. Next, the resulting composition is kneaded using a kneader such as, for example, a three roll mill, to obtain a fragmented gel structure.

Additionally, in another embodiment, the composition according to the present invention can be formed by blending a semi-crystalline polymer and a polyvinylpyrrolidone/α-olefin copolymer with an oil phase, heating to dissolve, then using any stirring apparatus to cool while stirring, to obtain a fragmented gel structure.

In a further embodiment, the composition according to the present invention can be formed by blending a semi-crystalline polymer and a polyvinylpyrrolidone/α-olefin copolymer with an oil phase, heating to dissolve, then passing the resulting composition through an extruder, to obtain a fragmented gel structure.

The composition for external application of the present invention is most preferably a composition that forms a paste or a cream at room temperature.

In the present invention, the expression “paste or cream form” refers to a non-solid, non-liquid form, i.e. a soft product, as is generally understood by those skilled in the art, and also includes fluid forms which are often expressed by other terms, such as gels. Additionally, paste or cream type compositions may be either oil-based or water-based, and can take the form of emulsion systems such as oil-in-water emulsion systems, water-in-oil emulsion systems, and non-aqueous gels or pastes, of which non-aqueous or water-in-oil emulsion systems are preferable.

In one form of the present invention, the property of being a “paste or cream form” can be expressed by specific measurable physicochemical properties. For example, the property of being a paste or a cream can be expressed by a specific range of viscosities, and for example, can be defined to be such as to have a measurable viscosity on Rotors Nos. 2, 3, 4 and 5 for 10 minutes at a shear rate of 200 s⁻¹ using a Rheomat RM180 (Rheometric Scientific), preferably being defined as having a viscosity of between 330 centipoise (lower limit, reading of 50 on Rotor No. 2) to 35000 centipoise (upper limit, reading of 30 on Rotor No. 5), and more preferably between 650 centipoise (lower limit, reading of 20 on Rotor No. 3) to 11800 centipoise (upper limit, reading of 50 on Rotor No. 4) at room temperature.

One embodiment of the composition of the present invention is characterized by comprising at least one type of semi-crystalline polymer having a melting point of at least 50° C., at least one type of polyvinylpyrrolidone/α-olefin copolymer having a melting point of at least 50° C., and an oil phase containing at least one type of non-volatile oil.

A. Semi-Crystalline Polymers

For the purposes of the present invention, the term “semi-crystalline polymer” has the same meaning as is understood by those skilled in the art, in particular polymers comprising an organic polymer backbone and a side chain (pendant chain), comprising a crystallizable part in a part of the side chain and/or the backbone, and an amorphous part in the backbone and exhibiting a first-order reversible phase change temperature, such as a melting temperature (solid-liquid transition). The term “polymers” is understood to mean, according to the present invention, compounds comprising at least two repeat units, such as at least 3 repeat units and further such as at least 10 repeat units. If the crystalline portion is a polymer backbone sequence, then the chemical properties of this crystalline sequence will differ from those of the amorphous sequence. In this case, a semi-crystalline polymer is a block polymer, and may, for example, be of diblock, triblock or multiblock type. The semi-crystalline polymer used in this invention is soluble or dispersible in the oil phase.

The semi-crystalline polymer or polymers of the composition of the invention comprise a number-average molecular mass (weight) Mn advantageously of at least 2,000, ranging from 2,000 to 800,000, preferably 3,000 to 500,000, for example from 4,000 to 150,000. More preferably, the number-average molecular mass is less than 100,000, in particular from 4,000 to 99,000. In addition, they may exhibit a number-average molecular mass of greater than 5,600, for example ranging from 5,700 to 99,000.

According to the invention, the semi-crystalline polymers are preferably soluble in the oil phase to at least 1% by weight at a temperature greater than their melting temperature. Apart from the crystallizable chains or blocks, the polymer sequences are amorphous. The term “crystallizable chain or block” is understood to mean, according to the present invention, a chain or block which, if it were alone, would change reversibly from the amorphous state to the crystalline state, according to whether the temperature is above or below the melting temperature. A chain within the meaning of the invention is a group of atoms which is in the pendent or side position with respect to the backbone of the polymer. A block is a group of atoms belonging to the backbone, a group constituting one of the repeat units of the polymer. Preferably, the polymer backbone of the semi-crystalline polymers is soluble in the oil phase.

Additionally, the semi-crystalline polymer of the present invention has a high melting point Mp, preferably such that 50° C.≦Mp≦120° C., more preferably such that 50° C.≦Mp≦100° C., and further such that 50° C.≦Mp≦70° C. This melting point is a first-order state change temperature. According to the invention, the melting point can be measured by any known method, such as with a differential scanning calorimeter (DSC). If the melting point is less than 50° C., the solidifying force is insufficient, which is undesirable in view of the high-temperature stability of the product. Additionally, if the melting point exceeds 120° C., high temperature processing becomes necessary for dissolution, which is undesirable.

For example, the crystallizable block or chain or blocks or chains of the semi-crystalline polymers preferably represent at least 30% of the total weight of each polymer such as at least 40%. The semi-crystalline polymers of the invention with crystallizable blocks are block or multiblock polymers. They can be obtained by polymerization of a monomer with reactive (or ethylenic) double bonds or by polycondensation. When the semi-crystalline polymers of the invention are polymers with crystallizable side chains, the latter are preferably in the random form.

The semi-crystalline polymers of the invention can be synthetic in origin, and in a preferred embodiment may not comprise a polysaccharide backbone.

According to the invention, the semi-crystalline polymer is chosen from block copolymers comprising at least one crystallizable block and at least one amorphous block, homopolymers and copolymers carrying at least one crystallizable side chain per repeat unit, and mixtures thereof.

The semi-crystalline polymers which can be used in the invention include:
(1) block copolymers of polyolefins with controlled crystallization, such as those for which the monomers are disclosed in EP-A-0 951 897;
(2) polycondensates of aliphatic or aromatic polyester type or aliphatic/aromatic copolyester type;
(3) homo- and copolymers carrying at least one crystallizable side chain and homo- or copolymers carrying, in the backbone, at least one crystallizable block, such as those disclosed in U.S. Pat. No. 5,156,911;
(4) homo- and copolymers carrying at least one crystallizable side chain with fluorinated group(s), such as disclosed in document WO-A-01/19333; and
(5) mixtures thereof.

In the last two cases (3 and 4), the crystallizable side chain or block or side chains or blocks are hydrophobic.

a) Semi-Crystalline Polymers with Crystallizable Side Chains

Mention may also be made of those defined in U.S. Pat. No. 5,156,911 and WO-A-01/19333.

These are homopolymers or copolymers comprising from 50 to 100% by weight of units resulting from the polymerization of one or more monomers carrying a crystallizable hydrophobic side chain.

These homo- or copolymers can have any nature provided that they exhibit the conditions indicated below, with for example the characteristic of being soluble or dispersible in the oil phase by heating above their melting temperature Mp. They can result for example:
from the polymerization, such as free radical polymerization, of one or more monomers with double bond(s) or ethylenic monomers reactive with respect to polymerization, namely with a vinyl, (meth)acrylic or allyl group; and
from the polycondensation of one or more monomers carrying coreactive groups (carboxylic or sulphonic acid, alcohol, amine or isocyanate groups), such as, for example, polyesters, polyurethanes, polyethers, polyureas or polyamides.

Generally, these polymers can be chosen from the homopolymers and copolymers resulting from the polymerization of at least one monomer with crystallizable chain(s) which can be represented by the formula (I): 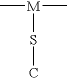
with M representing an atom of the polymer backbone, S representing a spacer, and C representing a crystallizable group.

The crystallizable chains “—S—C” can be aliphatic or aromatic and optimally fluorinated or perfluorinated. “S” can represent a linear or branched or cyclic (CH₂)ₙ or (CH₂CH₂O)ₙ or (CH₂O) group with n an integer ranging from 0 to 22. For instance, “S” is a linear group. According to another aspect of the invention, “S” and “C” are different.

When the crystallizable chains “—S—C” are hydrocarbonaceous aliphatic chains, they comprise hydrocarbonaceous alkyl chains with at least 11 carbon atoms and at most 40 carbon atoms, such as most 24 carbon atoms. They can be aliphatic chains or alkyl chains having at least 12 carbon atoms; according to one aspect of the invention, they are C₁₄-C₂₄ alkyl chains. When they are fluorinated or perfluorinated alkyl chains, they comprise at least 6 fluorinated carbon atoms and can have at least 11 carbon atoms, at least 6 carbon atoms of which are fluorinated.

Mention may be made, as non-limiting examples of semi-crystalline polymers with crystallizable chain(s), of those resulting from polymerization of one or more following monomers: saturated alkyl (meth)acrylates with the C₁₄-C₂₄ alkyl group; perfluoroalkyl (meth)acrylates with a C₁₁-C₁₅ perfluoroalkyl group; N-alkyl(meth)acrylamides with the C₁₄ to C₂₄ alkyl group, with or without a fluorine atom; vinyl esters with alkyl or perfluoro(alkyl) chains with the C₁₄ to C₂₄ alkyl group (with at least 6 fluorine atoms per one perfluoroalkyl chain); vinyl ethers with alkyl or perfluoro(alkyl) chains with the C₁₄ to C₂₄ alkyl group and at least 6 fluorine atoms per one perfluoroalkyl chain; C₁₄ to C₂₄ α-olefins such as, for example, octadecene; para-alkylstyrenes with an alkyl group comprising from 12 to 24 carbon atoms, and mixtures thereof.

The term “alkyl” is understood to mean, according to the present invention, a saturated group, for example a C₈-C₂₄ group, unless specifically mentioned, and also a C₁₄-C₂₄ group.

When the polymers result from a polycondensation, the crystallizable hydrocarbonaceous and/or fluorinated chains as defined above are carried by a monomer which can be a diacid, a diol, a diamine or a diisocyanate.

When the polymers which are the subject-matter of the invention are copolymers, they may additionally comprise from 0 to 50% of Y or Z groups resulting from the copolymerization:

α) of Y, which is a polar or nonpolar monomer or a mixture of the two:

When Y is a polar monomer, it is a monomer carrying polyoxyalkylenated groups (for example oxyethylenated and/or oxypropylenated groups); a hydroxyalkyl (meth)acrylate, such as hydroxyethyl acrylate; (meth)acrylamide; an N-alkyl(meth)acrylamide; an N,N-dialkyl(meth)acrylamide, such as, for example, N,N-diisopropylacrylamide; N-vinylpyrrolidone (NVP); N-vinylcaprolactam; or a monomer carrying at least one carboxylic acid group, such as (meth)acrylic acid, crotonic acid, itaconic acid, maleic acid or fumaric acid, or carrying a carboxylic acid anhydride group, such as maleic anhydride, and mixtures thereof. —When Y is a nonpolar monomer, it can be an ester of the linear, branched or cyclic alkyl (meth)acrylate type, a vinyl ester, an alkyl vinyl ether, an α-olefin, styrene or styrene substituted by a C₁-C₁₀ alkyl group, such as α-methylstyrene, or a macromonomer of the polyorganosiloxane type with vinyl unsaturation.
   β) of Z, which is a polar monomer or a mixture of polar monomers. In this case, Z has the same definition as the “polar Y” defined above.

For example, the semi-crystalline polymers with a crystallizable side chain are alkyl (meth)acrylate or alkyl(meth)acrylamide homopolymers with an alkyl group as defined above such as a C₁₄-C₂₄ alkyl group, copolymers of these monomers with a hydrophilic monomer can be different in nature from (meth)acrylic acid, such as N-vinylpyrrolidone or hydroxyethyl (meth)acrylate, and mixtures thereof.

b) Polymers Carrying, in the Backbone, at Least One Crystallizable Block

These are again preferably polymers which are soluble or dispersible in the oil phase by heating above their melting point Mp. These polymers can be block copolymers composed of at least two blocks of different chemical natures, one of which is crystallizable.

The following types of polymers are included:
Polymers defined in U.S. Pat. No. 5,156,911.
Block copolymers of olefin or of cycloolefin with a crystallizable chain, such as those resulting from the block polymerization of:
   cyclobutene, cyclohexene, cyclooctene, norbornene (that is to say, bicyclo[2,2,1]hept-2-ene), 5-methylnorbornene, 5-ethylnorbornene, 5,6-dimethylnorbornene, 5,5,6-trimethyinorbornene, 5-ethylidenenorbornene, 5-phenylnorbornene, 5-benzylnorbornene, 5-vinyinorbornene, 1,4,5,8-dimethano-1,2,3,4,4a,5,8a-octahydronaphthalene-, dicyclopentadiene or mixtures thereof, with
   ethylene, propylene, 1-butene, 3-methyl-1-butene, 1-hexene, 4-methyl-1-pentene, 1-octene, -1-decene, 1-eicosene or mixtures thereof;

These block copolymers may be for example copoly(ethylene/norbornene) blocks and (ethylene/propylene/ethylidenenorbornene) terpolymer blocks. Use may also be made of those resulting from the block copolymerization of at least 2 C₂-C₁₆ α-olefins for example C₂-C₁₂ α-olefins, such as those mentioned above, and further such as the block bipolymers of ethylene and 1-octene.
Copolymers exhibiting at least one crystallizable block, the remainder of the copolymer being amorphous (at ambient temperature).

These copolymers can, in addition, exhibit two crystallizable blocks of different chemical natures. Examples of copolymers are those which have, at ambient temperature, both a crystallizable block and a both hydrophobic and lipophilic amorphous block which are sequentially distributed; mention may be made, for example, of the polymers having one of the following crystallizable blocks and one of the following amorphous blocks:
Block crystallizable by nature: a) polyester, such as poly(alkylene terephthalate)s, b) polyolefin, such as polyethylenes or polypropylenes.
Amorphous and lipophilic block, such as: amorphous polyolefins or copoly(olefin)s, for example poly(isobutylene), hydrogenated polybutadiene or hydrogenated poly(isoprene).

Mention may be made, as suitable non-limiting examples of such copolymers with a crystallizable block and with an amorphous block, of:
α) poly(ε-caprolactone)-b-poly(butadiene) block copolymers, such as used hydrogenated, such as those described in the article, “Melting Behavior of poly(ε-caprolactone)-block-polybutadiene copolymers”, by S. Nojima, Macromolecules, 32, 3727-3734 (1999);
β) block or multiblock hydrogenated poly(butylene terephthalate)-b-poly(isoprene) block copolymers, cited in the article, “Study of Morphological and Mechanical Properties of PP/PBT”, by B. Boutevin et al., Polymer Bulletin, 34, 117-123 (1995);
γ) the poly(ethylene)-b-copoly(ethylene/propylene) block copolymers cited in the articles, “Morphology of Semi-Crystalline Block Copolymers of ethylene-(ethylene-alt-propylene)”, by P. Rangarajan et al., Macromolecules, 26, 4640-4645 (1993) and “Polymer Aggregates with Crystalline Cores: the System poly(ethylene)-poly(ethylene-propylene-)”, P. Richter et al., Macromolecules, 30, 1053-1068 (1997); and
δ) the poly(ethylene)-b-poly(ethylethylene) block copolymers cited in the general article, “Crystallization in Block Copolymers”, by I. W. Hamley, Advances in Polymer Science, vol. 148, 113-137 (1999).

The semi-crystalline polymers of the composition of the invention may or may not be (partially) crosslinked provided that the degree of crosslinking is not too harmful to their dissolution or dispersion in the oil phase by heating above their melting temperature. The crosslinking can then be chemical crosslinking, by reaction with a multifunctional monomer during the polymerization. It can also be physical crosslinking, which can then be due either to the establishment of bonds of hydrogen or dipolar type between groups carried by the polymer, such as, for example, dipolar interactions between carboxylate ionomers, these interactions being low in degree and carried by the backbone of the polymer, or to phase separation between the crystallizable blocks and the amorphous blocks carried by the polymer.

The semi-crystalline polymers of the composition according to the invention can, for example, be not crosslinked.

According to an embodiment of the invention, the polymer can be chosen from the copolymers resulting from the polymerization of at least one monomer with a crystallizable chain, chosen from saturated C₁₄-C₂₄ alkyl (meth)acrylates; C₁₁-C₁₅ perfluoroalkyl (meth)acrylates; N-(C₁₄-C₂₄ alkyl)(meth)acrylamides, with or without at least one fluorine atom; vinyl esters with C₁₄ to C₂₄ alkyl or perfluoroalkyl chains; vinyl ethers with C₁₄ to C₂₄ alkyl or perfluoroalkyl chains; C₁₄ to C₂₄ α-olefins; or para-alkylstyrenes with an alkyl group comprising from 12 to 24 carbon atoms, with at least one optionally fluorinated C₁ to C₁₀ monocarboxylic acid ester or amide, which can be represented by the following formula: 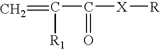
wherein R₁ is chosen from H and CH₃, R is chosen from optionally fluorinated C₁-C₁₀ alkyl groups, and X is chosen from O, NH and NR₂, where R₂ represents an optionally fluorinated C₁-C₁₀ alkyl group.

According to an embodiment of the invention, the polymer can result from a monomer with a crystallizable chain chosen from saturated C₁₄-C₂₂ alkyl (meth)acrylates.

Mention may be made, as non-limiting examples of structuring semi-crystalline polymers which can be used in the composition according to the invention, of the Intelimer® products from Landec described in the brochure “Intelimer® polymers”, Landec IP22 (Rev. 4-97). These polymers are in the solid form at ambient temperature. They carry crystallizable side chains and exhibit the above formula (I).

Use may be made of the semi-crystalline polymers with a melting point of at least 50° C. obtained by copolymerization of behenyl acrylate and of acrylic acid or of NVP as disclosed in U.S. Pat. No. 5,519,063, such as those described in the polymer preparation Examples 1 and 2 below, with melting temperatures of 60° C. and 58° C., respectively.

In one example, the semi-crystalline polymer does not comprise a carboxyl group.

Use may be made also of the semi-crystalline polymers described in the Application US2001/0018484.

Preferably, the semi-crystalline polymer used in the present application is an homopolymers obtained by polymerization of behenyl acrylate, specially the polymer named “Intelimer IPA-13.6” of Landec Coporation. This polymer has a melting temperature of 66° C.

The composition of the present invention preferably contains the semi-crystalline polymers in an amount of 0.1-14% by weight, more preferably 1-5% by weight with respect to the total weight of the composition, although other amounts are possible.

B. Vinylpyrrolidone/α-Olefin Copolymer

The preferred polyvinylpyrrolidone/α-olefin copolymers used in the present invention have a melting point of at least 50° C., are lipophilic, and can be expressed by the following formula: 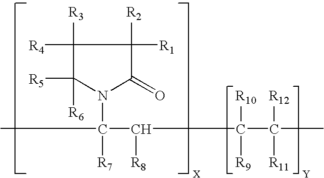
wherein the radicals R₁ to R₁₂ represent, independently one from another, a C₁₀ to C₄₀ linear or branched saturated alkyl group or a hydrogen atom, at least one of the aforementioned R₁ to R₁₂ being different from a hydrogen atom. The value Y may be equal to 0; x must not be equal to 0.

Preferably, at least one of the radicals which are not hydrogen atoms contains 14 to 32 carbon atoms, more preferably 28 to 32 carbon atoms.

Among the alkyl groups having 10 to 40 carbon atoms, it is possible to cite as examples pentadecyl, hexadecyl, heptadecyl, octadecyl, nonadecyl, eicosyl, docosyle and triacontyl groups.

The average molecular mass of the polymer of the present invention is preferably 5,000 to 30,000, in particular, 6,000 to 20,000.

In a preferred embodiment, Y is equal to 0 and R₂ to R₅ represent hydrogen atoms. Preferably at least one of the radicals aside from the hydrogens have 14 to 32 carbon atoms.

Inn another embodiment, Y is not equal to 0. The radicals R₁ to R₉ and R₁₁ to R₁₂ preferably represent hydrogen atoms. Preferably, R₁₀ has 14 to 32 carbon atoms, and the ratio x:y is from 1:5 to 5:1.

Among the commercial products which can be used in the present invention, it is possible to cite as an example Antarons™ such as Antaron WP660 whose CTFA moniker is tricontanyl-PVP.

The composition of the present invention preferably comprises the vinylpyrrolidone/α-olefin copolymer in an amount of 0.1-14% by weight, more preferably 0.3-3.5% by weight with respect to the total weight of the composition, although other amounts can be used.

The total amount of the semi-crystalline polymer and the vinylpyrrolidone/α-olefin copolymer should preferably be 1-15% by weight, especially 2-5% by weight with respect to the total weight of the composition; furthermore, the amount of the crystallizable polymer with respect to the amount of the vinylpyrrolidone/α-olefin copolymer should preferably be such that their ratio is 0.1-10, preferably 1-5.

C. Oil Phase

The oil phase of the composition according to the present invention comprises at least one non-volatile oil.

Non-volatile oils can be any oils such as are generally used in cosmetics, with the exception of the volatile oils described below. Preferably, they are oils or polyesters bearing at least two ester groups in the molecule, which are liquid at room temperature (25° C.) and atmospheric pressure (760 mmHg).

More preferably, they are chosen from among the following:
Hydrocarbons with a high content of triglycerides composed (C₄ to C₂₄) fatty acids and glycerin, the fatty acids of which can be linear or branched and saturated or unsaturated. Specific examples include triglycerides of heptanoic acid, triglycerides of octanoic acid, wheat germ oil, maize oil, sunflower oil, karate oil, castor oil, almond oil, macadamia oil, apricot oil, soybean oil, rapeseed oil, cottonseed oil, alfalfa oil, poppy oil, potimarron oil, sesame oil, pumpkin oil, avocado oil, walnut oil, grapeseed oil, blackcurrant seed oil, evening primrose oil, millet oil, barley oil, quinoa oil, olive oil, rye oil, safflower oil, candlenut oil, passionflower oil, musk rose oil, coriander seed oil; or Glyceryl tri-(caprylate/caprate) such as those sold by Cognis under the name Myritol 318 or those sold by Stearineries Dubois under the names Triglycerides C8-C10 70/30.
Esters of fatty alcohols such as di-isostearyl malate, tri-isocetyle citrate and tridecyl trimellitate.
Polyolesters, for example, propylene glycol dioctanoate, neopentyl glycol diheptanoate and diethylene glycol diisononoate.
Esters of pentaerythritol, such as pentaerythrityl tetraisostearate.
Mixtures thereof.

As non-volatile oils, it is possible to use also lipophilic liquid UV absorbents such as ethylhexyl methoxycinnamate, commercial product Parsol MCX (Roche Vitamins), commercial product Escalol 557L (IPS) or Uvinul MC80 (BASF).

The non-volatile oil of the oil phase may be provided in an amount of 1-99% by weight, preferably 5-80% by weight with respect to the total weight of the oil phase.

The present invention may further contain volatile oils. The volatile oils which are used should preferably have a sufficiently high flash point.

As these volatile oils, cyclic or linear hydrocarbonaceous oils and/or silicone oils may be selected.

Among silicone oils, the following may be used, either alone or in a mixture.
Volatile cyclic silicones having 3 to 8, preferably 4 to 6 silicon atoms, for example, cyclotetradimethylsiloxane, cyclopentadimethylsiloxane or cyclohexadimethylsiloxane, and cyclocopolymers of dimethylsiloxane/methylalkylsiloxane type, such as Silicon FZ 3109 of Union Carbide, which is a dimethylsiloxane/methyloctylsiloxane cyclocopolymer.
Volatile linear silicones having 2 to 9 silicon atoms, for example, hexamethyldisiloxane or low-viscosity (1 cSt) PDMS. Further examples include alkyltrisiloxanes such as hexylheptamethyltrisiloxane or octylheptamethyltrisiloxane.

The oil phase is preferably of an amount such that the total weight of the semi-crystalline polymer and vinylpyrrolidone/α-olefin copolymer with respect to the oil phase is such that their ratio is 0.01-1.5, preferably 0.02-0.6.

The composition of the present invention may also comprise an emulsifier and an aqueous phase.

The emulsifiers used in the present invention may be of any type, and preferably are capable of being used in the cosmetic and dermatological fields. Those skilled in the art will be capable of selecting the appropriate types and amounts of emulsifiers so as not to substantially change the favorable properties of the composition of the present invention, in view of this disclosure.

As emulsifiers that may be used for the preparation of the W/O emulsions, mention may be made, for example, of the alkyl esters or ethers of sorbitan, of glycerol or of sugars; silicone surfactants. As examples of silicon surfactants, may be cited for instance dimethicone copolyols such as the product sold under the name KF6017 by the company Shin-Etsu, the mixture of polyglyceryl-4 isostearate/cetyl dimethicone copolyol/hexyl laurate, sold under the name Abil WE 09 by the company Goldschmidt, the mixture of cyclomethicone and of dimethicone copolyol, sold under the names DC 5225 C and DC 3225 C by the company Dow Corning or sold under the name Abil EM 97 by the company Goldschmidt, and alkyldimethicone copolyols such as the laurylmethicone copolyol sold under the name “Dow Corning 5200 Formulation Aid” by the company Dow Corning and the cetyldimethicone copolyol sold under the name Abil EM 90 by the company Goldschmidt. It is also possible to add thereto one or more co-emulsifiers, which may be chosen advantageously from the group comprising esters of a branched-chain fatty acid and of polyol, and especially esters of a branched-chain fatty acid and of glycerol and/or of sorbitan, and, for example, polyglyceryl isostearate, such as the product sold under the name Isolan GI 34 by the company Goldschmidt, sorbitan isostearate, such as the product sold under the name Arlacel 987 by the company ICI, and the isostearate of sorbitan and of glycerol, such as the product sold under the name Arlacel 986 by the company ICI, and mixtures thereof.

For the O/W emulsions, examples of emulsifiers that may be mentioned include non-ionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate, and mixtures thereof such as, for example, the mixture of glyceryl stearate and of PEG-100 stearate, sold under the name Aracel 165 by the company Uniqema.

If the composition of the present invention includes an aqueous phase, the aqueous phase may include, aside from water, solvents such as primary alcohols containing 1-6 carbon atoms, such as ethanol, or polyols such as butylenes glycol, glycerin, sorbitol, hexylene glycol, propylene glycol or isopropylene glycol, or sugars such as glucose or fructose.

The composition of the present invention may further contain additives such as those conventionally used in the cosmetic or dermatological fields, within a range such as not to (overly) adversely affect the advantageous properties of the composition according to the present invention. Examples of such additives include, for example, oil-based raw materials other than the above-mentioned oils, such as waxes, gelling agents aside from the above-mentioned polymers, particularly semi-crystalline waxes having a melting point less than 50° C., antioxidants, fragrances, essential oils, preservatives, lipophilic or hydrophilic cosmetic active agents, humectants, fillers (organic powders or mineral particles), vitamins, dyes, essential fatty acids, sphingolipids, self-tanning agents such as DHA, sunscreens and the like. These additives can be included in the composition in an amount, for example, of 0-10 wt %.

While the composition of the present invention can be manufactured by various methods, in order to prevent solidification due to the gelling agent, the gel structure of the oil phase should be destroyed or the progress in the gelification suppressed by, e.g., stirring during gelification of the oil phase to form a fine gel structure.

Preferably, the composition of the present invention is produced by the following manufacturing method:
After forming a solid composition in the same manner as in the manufacturing method for a stick or compact type product, it is kneaded by shear means such as a three-roll mill.
After heating and melting the composition including a gelling agent, appropriate agitation means are used to cool while agitating.
After heating and melting the composition including a gelling agent, it is passed through an extruder.

For example, the methods described in EP 0 755 668 and EP 0 745 376 may be used.

Additionally, other means which are publicly known in the field may be used to form a fine gel structure. For example, during gelification, means such as keeping the temperature less than the temperature at which it completely melts, putting into an air-dissolved state, causing fine motion such as with ultrasonic vibrations, or performing cavitation can be used either alone or in combination.

The composition of the present invention can be used as a cosmetic composition or an external agent for application to the skin, and is especially suited to be a cosmetic composition for protection, care, makeup or makeup removal and/or cleansing of the skin (including scalp), hair, nails and/or mucous membrane. Especially, the composition may constitute a make-up remover or a foundation.

### EXAMPLES

The invention will now be illustrated by the following non-limiting examples. Where not otherwise indicated, % refers to % by weight.

### I) Composition Formulation Examples

In the following Examples 3-5, gelification of the various types of oil phases was attempted according to the following methods, and destruction of the gel structures was attempted, to prepare the compositions:
1. Heat oils to 75° C. while stirring with a paddle.
2. Add polymer and stir for 20 minutes at 75° C.
3. Store bulk in incubator of 25° C. for 12 hours.
4. Pass once through three roll mill.

The properties and viscosities of the resulting compositions were measured at various temperatures.

### Example 1

Gelification of Volatile Oils (Reference Example)

| | A1 | A2 | A3 | A4 | A5 | B1 | B2 | B3 | B4 | B5 |
|---|---|---|---|---|---|---|---|---|---|---|
| Semi-Crystalline Polymer¹ | 6% | 3% | — | — | — | 6% | 3% | — | — | — |
| (Melting Point: 49° C.) | | | | | | | | | | |
| Vinylpyrrolidone/α-Olefin | — | 3% | 6% | 3% | — | — | 3% | 6% | 3% | — |
| Copolymer² | | | | | | | | | | |
| (Melting Point: 63° C.) | | | | | | | | | | |
| Semi-Crystalline Polymer³ | — | — | — | 3% | 6% | — | — | — | 3% | 6% |
| (Melting Point: 66° C.) | | | | | | | | | | |
| Cyclopentadimethylsiloxane⁴ | 94% | 94% | 94% | 94% | 94% | — | — | — | — | — |
| Isododecane⁵ | — | — | — | — | — | 94% | 94% | 94% | 94% | 94% |

| | |
|---|---|
| Aspect | Not gelified |

¹Homopolymer of stearyl acrylate (Intelimer IPA-13.1 of Landec)
²Antaron WP-660
³Homopolymer of behenyl acrylate (Intelimer IPA-13.6 of Landec)
⁴DC 245 Fluid (Dow-Corning)
⁵Permethyl 99A (Permethyl)

### Example 2

### Gelification of Apricot Seed Oil (Present Invention)

| | C1 | C2 | C3 | C4 | C5 |
|---|---|---|---|---|---|
| | Comparative | Comparative | Comparative | Present | Comparative |
| | Product | Product | Product | Invention | Product |
| Semi-Crystalline | 6% | 3% | — | — | — |
| Polymer¹ | | | | | |
| (Melting Point: | | | | | |
| 49° C.) | | | | | |
| Vinylpyrrolidone/α-Olefin | — | 3% | 6% | 3% | — |
| Copolymer² | | | | | |
| (Melting Point: | | | | | |
| 63° C.) | | | | | |
| Semi-Crystalline | — | — | — | 3% | 6% |
| Polymer³ | | | | | |
| (Melting Point: | | | | | |
| 66° C.) | | | | | |
| Apricot Kernel Oil⁶ | 94% | 94% | 94% | 94% | 94% |
| Aspect at 25° C. (after | Gel | Gel | Gel | Gel | Gel |
| 24 h) | | | | | |
| Aspect at 45° C. (after | Liquid | Liquid | Liquid | Gel | Slight gel |
| 24 h) | | | | | |
| Viscosity at 25° C. | 44.7 on | 51.7 on | 34.7 on | 26.2 on | 19.7 on |
| (after 24 h)* | M3 | M2 | M2 | M3 | M3 |
| | 1712 cps | 349 cps | 216 cps | 915 cps | 661 cps |

| | | | |
|---|---|---|---|
| Viscosity at 45° C. | Too fluid, not measured. | 13.5 on | 8.87 on |
| (after 24 h)* | | M3 | M3 |
| | | 450 cps | 283 cps |

*The viscosity results are the viscosities measured at the respective temperatures after leaving at the various temperatures for 24 hours. The top portion gives the readings on the viscometer RM180 and the bottom portion gives the values after conversion to units of centipoise.
⁶Crude Apricot Almond Oil MP301 (Safic-Alcan)

This table shows that a polymer having a melting temperature of at least 50° C. provides a preferred composition.

### Example 3

Gelification of Caprylic/Capric Acid Triglyceride (Present Invention)

| | D1 | D2 | D3 | D4 | D5 |
|---|---|---|---|---|---|
| | Comparative | Comparative | Comparative | Present | Comparative |
| | Product | Product | Product | Invention | Product |
| Semi-Crystalline | 6% | 3% | — | — | — |
| Polymer¹ | | | | | |
| (Melting Point: | | | | | |
| 49° C.) | | | | | |
| Vinylpyrrolidone/α-Olefin | — | 3% | 6% | 3% | — |
| Copolymer² | | | | | |
| (Melting Point: | | | | | |
| 63° C.) | | | | | |
| Semi-Crystalline | — | — | — | 3% | 6% |
| Polymer³ | | | | | |
| (Melting Point: | | | | | |
| 66° C.) | | | | | |
| Caprylic/Capric Acid | 94% | 94% | 94% | 94% | 94% |
| Triglyceride⁷ | | | | | |
| Aspect at 25° C. (after | Gel | Gel | Gel | Gel | Gel |
| 24 h) | | | | | |
| Aspect at 45° C. (after | Liquid | Liquid | Liquid | Gel | Slight gel |
| 24 h) | | | | | |
| Viscosity at 25° C. | 41.4 on | 15.0 on | 35.2 on | 29.6 on | 22.2 on |
| (after 24 h)* | M3 | M3 | M2 | M3 | M3 |
| | 1542 cps | 483 cps | 216 cps | 1076 cps | 746 cps |

| | | | |
|---|---|---|---|
| Viscosity at 45° C. | Too fluid, not measured | 14.1 on | 12.8 on |
| (after 24 h)* | | M3 | M3 |
| | | 450 cps | 417 cps |

⁷Miglyol 318 (Cognis)

Example 1 (Reference Example) shows that volatile oils do not gelify even when used in conjunction with a semi-crystalline polymer and a polyvinylpyrrolidone/α-olefin copolymer. Additionally, in Examples 2-3, no matter which non-volatile oil is used, if one of the semi-crystalline polymer and the polyvinylpyrrolidone/α-olefin copolymer is removed, the stability of the gel is lost at high temperatures, it becomes liquid (Compositions C1-C3 and D1-D3), or does not have sufficient viscosity (Compositions C5 and D5), but when using both the semi-crystalline polymer and the polyvinylpyrrolidone/α-olefin copolymer (Composition C4 and D4), a stable gel structure is obtained even at high temperatures.

### III) Cosmetic Composition Formulation Example

### Example 4

Gel-type Cleansing Oil

Production Method:
1. The oil phase, surfactant and aqueous phase were heated to 75° C. while agitating with a paddle.
2. The polymers other than silica dimethylsilylate were added and completely dissolved.
3. Silica dimethylsilylate was dispersed with a paddle and completely soaked into the bulk.
4. The paddle was changed to a Rayneri, and the bulk was dispersed until smooth at 75° C.
5. The bulk was let stand at room temperature.

6. After 24 hours, the result was passed through a three roll mill.

| | E1 | E2 | E3 | E4 | E5 | E6 |
|---|---|---|---|---|---|---|
| | Comp | Pres | Comp | Comp | Comp | Comp |
| | Prod | Inv | Prod | Prod | Prod | Prod |
| Oil Phase | | | | | | |
| Caprylic/Capric | 38.5% | 38.5% | 39.5% | 38.5% | 38.5% | 41.5% |
| Triglyceride | | | | | | |
| Isohexadecane | 15% | 15% | 15% | 15% | 15% | 15% |
| Jojoba Seed Oil⁸ | 10.5% | 10.5% | 10.5% | 10.5% | 10.5% | 10.5% |
| Apricot Kernel Oil | 10.5% | 10.5% | 10.5% | 10.5% | 10.5% | 10.5% |
| Surfactant | | | | | | |
| POE-20 Glyceryl | 16% | 16% | 16% | 16% | 16% | 16% |
| Triisostearate⁹ | | | | | | |
| Aqueous Phase | | | | | | |
| Water | 0.5% | 0.5% | 0.5% | 0.5% | 0.5% | 0.5% |
| Polymer | | | | | | |
| Silica Dimethylsilylate¹⁰ | 6% | 6% | 6% | 6% | 6% | 6% |
| Semi-Crystalline Polymer¹ | 2% | — | — | — | — | — |
| (Melting Point: 49° C.) | | | | | | |
| Semi-Crystalline Polymer³ | — | 2% | 2% | 2% | 2% | — |
| (Melting Point: 66° C.) | | | | | | |
| Tricontanyl PVP¹¹ | 1% | 1% | — | — | — | — |
| Vp/Hexadecene | | | | | | |
| Copolymer¹² | — | — | — | 1% | — | — |
| Vp/Eicosene Copolymer¹³ | — | — | — | — | 1% | — |
| Aspect 24 h after | Pale yellow opalescent cream | | | | | Pale |
| production | | | | | | yellow |
| | | | | | | transparent |
| | | | | | | fluid |
| Viscosity Roller: | M3: 28.0 | M3: 39.7 | M2: 38.8 | M2: 49.3 | M2: 55.5 | M2: 35.6 |
| Reading | 983 cps | 1500 cps | 248 cps | 324 cps | 382 cps | 221 cps |
| Centipoise | | | | | | |
| Centrifugation (1 h, g = 900) | Separat. | Stable | Separat. | Separat. | Separat. | Separat. |
| Stability 2 months after | Unstable | Stable | Stable | Unstable | Unstable | Unstable |
| production | | | | | | |

⁸Pure Golden Jojoba Oil (Desert Whale)
⁹Emalex GWIS-320 EX (Nihon Emulsion)
¹⁰Aerosil R 972 (Degussa)
¹¹Antaron WP-660; melting point 63° C.; aspect: solid flakes
¹²Antaron V-216; aspect: viscous fluid
¹³Antaron V-220; melting point 35° C.; aspect: waxy flakes

### Example 5

Cream Foundation

| | F1 | F2 | F3 | F4 | F5 | F6 | F7 | F8 | F9 |
|---|---|---|---|---|---|---|---|---|---|
| | Comp | Comp | Comp | Comp | Comp | Pres | Pres | Pres | Comp |
| | Prod | Prod | Prod | Prod | Prod | Inv | Inv | Inv | Prod |
| Oil Phase | | | | | | | | | |
| Cyclopentasiloxane | 14.4% | 14.4% | 14.4% | 14.4% | 14.4% | 14.4% | 14.4% | 14.4% | 14.4% |
| Isododecane | 12% | 12% | 12% | 12% | 12% | 12% | 12% | 12% | 12% |
| Ethylhexyl | 7.5% | 7.5% | 7.5% | 7.5% | 7.5% | 7.5% | 7.5% | 7.5% | 7.5% |
| Methoxycinnamate | | | | | | | | | |
| Preservative | 0.1% | 0.1% | 0.1% | 0.1% | 0.1% | 0.1% | 0.1% | 0.1% | 0.1% |
| Surfactant | | | | | | | | | |
| BIS-PEG/PPG-14/14 | 1% | 1% | 1% | 1% | 1% | 1% | 1% | 1% | 1% |
| Dimethicone¹⁴ | | | | | | | | | |
| PEG-10 | 3% | 3% | 3% | 3% | 3% | 3% | 3% | 3% | 3% |
| Dimethicone¹⁵ | | | | | | | | | |
| Polymer | | | | | | | | | |
| Semi-Crystalline | — | 1% | 2% | 3% | 4% | — | — | — | — |
| Polymer¹ | | | | | | | | | |
| Melting Point: | | | | | | | | | |
| 49° C. | | | | | | | | | |
| Semi-Crystalline | — | — | — | — | — | 1% | 2% | 3% | 4% |
| Polymer³ | | | | | | | | | |
| Melting Point: | | | | | | | | | |
| 66° C. | | | | | | | | | |
| Tricontanyl PVP | 4% | 3% | 2% | 1% | — | 3% | 2% | 1% | — |
| Aqueous Phase | | | | | | | | | |
| Water | bal | bal | bal | bal | bal | bal | bal | bal | bal |
| Magnesium Sulfate | 0.7% | 0.7% | 0.7% | 0.7% | 0.7% | 0.7% | 0.7% | 0.7% | 0.7% |
| Preservative | 1.1% | 1.1% | 1.1% | 1.1% | 1.1% | 1.1% | 1.1% | 1.1% | 1.1% |
| Pigments, Fillers | | | | | | | | | |
| Nano Titanium | 5.74% | 5.74% | 5.74% | 5.74% | 5.74% | 5.74% | 5.74% | 5.74% | 5.74% |
| Dioxide treated with | | | | | | | | | |
| Aluminum | | | | | | | | | |
| Hydroxide, | | | | | | | | | |
| Dimethicone and | | | | | | | | | |
| Methicone¹⁶ | | | | | | | | | |
| Iron Oxides treated | 1.29% | 1.29% | 1.29% | 1.29% | 1.29% | 1.29% | 1.29% | 1.29% | 1.29% |
| with C9-15 | | | | | | | | | |
| Fluoroalcohol | | | | | | | | | |
| Phosphates | | | | | | | | | |
| Titanium Dioxide | 7.71% | 7.71% | 7.71% | 7.71% | 7.71% | 7.71% | 7.71% | 7.71% | 7.71% |
| treated with C9-15 | | | | | | | | | |
| Fluoroalcohol | | | | | | | | | |
| Phosphates | | | | | | | | | |
| Nylon-12¹⁷ | 6% | 6% | 6% | 6% | 6% | 6% | 6% | 6% | 6% |
| Silica¹⁸ | 3% | 3% | 3% | 3% | 3% | 3% | 3% | 3% | 3% |
| UV Agents, Vitamins | | | | | | | | | |
| Terephtalylidene | 2.1% | 2.1% | 2.1% | 2.1% | 2.1% | 2.1% | 2.1% | 2.1% | 2.1% |
| Dicamphor Sulfonic | | | | | | | | | |
| Acid Solution | | | | | | | | | |
| Triethanolamine | 0.37% | 0.37% | 0.37% | 0.37% | 0.37% | 0.37% | 0.37% | 0.37% | 0.37% |
| Tocopherol | 0.08% | 0.08% | 0.08% | 0.08% | 0.08% | 0.08% | 0.08% | 0.08% | 0.08% |
| Viscosity 24 h after | | | | | | | | | |
| production | | | | | | | | | |
| Roller 3 Reading | 20.5 | 32.4 | 43.1 | 58.3 | 52.6 | 49.0 | 52.3 | 74.7 | 34.9 |
| Centipoise | 712 | 1161 | 1672 | 2254 | 2048 | 1881 | 2006 | 2966 | 1288 |
| Centrifugation | 1 mm | stable | stable | stable | stable | stable | stable | stable | slight |
| (1 h, g = 900) | sep | | | | | | | | sep |
| Stability after 2 | | | | | | | | | |
| months | | | | | | | | | |
| 4° C. | stable | stable | stable | stable | stable | stable | stable | stable | stable |
| 25° C. | stable | stable | stable | stable | stable | stable | stable | stable | stable |
| 37° C. | unstbl | unstbl | unstbl | unstbl | unstbl | stable | stable | stable | stable |
| 45° C. | unstbl | unstbl | unstbl | unstbl | unstbl | slight | stable | stable | stable |
| | | | | | | unstbl | | | |

¹⁴Abil EM 97 (Degussa)
¹⁵KF-6017 (Shin-Etsu Chemical)
¹⁶Mibrid Powder SAS-TTO-S-3 (16%) (Miyoshi Kasei)
¹⁷Orgasol 2002 Extra D Nat Cos (Atochem)
¹⁸Sunsphere H-51 (Asahi Glass)

Method:

| Phase A1 | |
|---|---|
| BIS-PEG/PPG-14/14 Dimethicone | 1% |
| PEG-10 Dimethicone | 3% |
| Cyclopentasiloxane | 8.4% |
| Isododecane | 12% |
| Ethylhexyl Methoxycinnamate | 7.5% |
| Preservative | 0.1% |
| Phase A2 | |
| Semi-Crystalline Polymer (Intelimer IPA-13.6) | 3% |
| Melting Point 66° C. | |
| Tricontanyl PVP | 1% |
| Phase A3 | |
| Nano Titanium Dioxide treated with Aluminum | 5.74% |
| Hydroxide, Dimethicone and Methicone | |
| Phase A4 | |
| Iron Oxides treated with C9-15 Fluoroalcohol | 1.29% |
| Phosphates | |
| Titanium Dioxide treated with C9-15 Fluoroalcohol | 7.71% |
| Phosphates | |
| Cyclopentasiloxane | 6% |
| Phase A5 | |
| Nylon-12 | 6% |
| Silica | 3% |
| Phase A6 | |
| Tocopherol | 0.08% |
| Phase B | |
| Water | balance to 100% |
| Magnesium Sulfate | 0.7% |
| Preservative | 1.1% |
| Phase C | |
| Terephthalylidene Dicamphor Sulfonic Acid Solution | 2.1% |

(Preparation)
Phase A4: The pigment and cyclopentasiloxane were passed three times through a three roll mill.
Phase B: B was completely dissolved and adjusted to 50° C.
(Production)
1. Phase A1 was poured into a main beaker, and completely dissolved at room temperature using a magnetic stirrer.
2. Phase A2 was added and dissolved at 60-70° C.
3. Phase A3 was added and dispersed with a Mizuho homogenizer (10 minutes).
4. Phase A4 was added and dispersed (20-30 minutes).
5. Phase A5 was added and dispersed (5 minutes).
6. Phase B was added and emulsified at 50° C. (5 minutes).
7. Cooled to room temperature, and Phases A6 and C were added.
8. Changed to a paddle mixer, and stirred for 10-20 minutes to remove bubbles.

The invention compositions are useful for, e.g., in particular for caring for, protecting and/or making up, body or facial skin, the eyelashes and/or the lips, and/or for haircare, and thus for general application to keratin materials.

The above written description of the invention provides a manner and process of making and using it such that any person skilled in this art is enabled to make and use the same, this enablement being provided in particular for the subject matter of the appended claims, which make up a part of the original description and including a composition having a paste or cream form at 25° C., comprising at least one semi-crystalline polymer having a melting point of at least 50° C., at least one polyvinylpyrrolidone/α-olefin copolymer having a melting point of at least 50° C., and an oil phase comprising at least one non-volatile oil. Similarly enabled is a method for producing a composition, comprising:
blending at least one semi-crystalline polymer having a melting point of at least 50° C., at least one polyvinylpyrrolidone/α-olefin copolymer having a melting point of at least 50° C., and an oil phase comprising at least one non-volatile oil;
gelifying said oil phase; and
applying a shear force after gelification and/or during gelification to obtain a fragmented gel structure.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition having a paste or cream form at 25° C., comprising at least one semi-crystalline polymer having a melting point of at least 50° C., at least one polyvinylpyrrolidone/α-olefin copolymer having a melting point of at least 50° C., and an oil phase comprising at least one non-volatile oil.

2. A composition according to claim 1, whose viscosity at room temperature is from 330 centipoise to 35,000 centipoise.

3. A composition according to claim 1, wherein said paste or cream form is provided due to a gel structure being destroyed after gelification of the oil phase, or a progress of gelification being suppressed during gelification of the oil phase.

4. A composition according to claim 1, wherein the semi-crystalline polymer has an organic structure with a crystalline side chain and/or crystalline polymer backbone block, and has a number average molecular weight of at least 2,000.

5. A composition according to claim 1, wherein the semi-crystalline polymer is selected from the group consisting of homopolymers and copolymers formed by polymerization of at least one monomer containing a crystalline chain expressed by the formula (I): 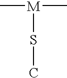
where M represents an atom of the polymer backbone, S represents a spacer, and C represens a crystallizable group, and the crystallizable chain “—S—C” represents an alkyl chain comprising at least 11 carbon atoms which can be fluorinated or perfluorinated, and mixtures thereof.

6. A composition according to claim 1, wherein the semi-crystalline polymer has a melting point of 50-120° C., and a number average molecular weight of 2,000 to 800,000.

7. A composition according to claim 1, wherein the semi-crystalline polymer is a homopolymer of behenyl acrylate.

8. A composition according to claim 1, wherein the semi-crystalline polymer is present in the composition in an amount of 0.1-14% by weight with respect to the total weight of the composition.

9. A composition according to claim 1, wherein the polyvinylpyrrolidone/α-olefin copolymer has a melting point of 50-70° C.

10. A composition according to claim 1, wherein the polyvinylpyrrolidone/α-olefin copolymer is a polymer represented by the following formula: 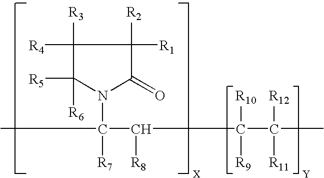
wherein the radicals R₁ to R₁₂ represent, independently one from another, a C₁₀ to C₄₀ linear or branched saturated alkyl group or a hydrogen atom, at least one of the aforementioned R₁ to R₁₂ being different from a hydrogen atom, X and Y are integers, and the value Y may be equal to 0 but X must not be equal to 0.

11. A composition according to claim 1, wherein the polyvinylpyrrolidone/α-olefin copolymer is present in the composition in an amount of 0.1-14% by weight with respect to the total weight of the composition.

12. A composition according to claim 1, wherein the non-volatile oil is present in the composition in an amount of 1-99% by weight with respect to the total weight of the composition.

13. A composition according to claim 1, which is a water-in-oil emulsion or non-aqueous.

14. A composition according to claim 1, which is a cosmetic composition.

15. A composition according to claim 1, wherein the semi-crystalline polymer is selected from the group consisting of homopolymers and copolymers formed by polymerization of at least one monomer containing a crystalline chain expressed by the formula (I): 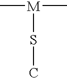
where M represents an atom of the polymer backbone, S represents a spacer, and C represens a crystallizable group, and the crystallizable chain “—S—C” represents an alkyl chain comprising at least 11 carbon atoms which can be fluorinated or perfluorinated, and mixtures thereof,
and wherein the polyvinylpyrrolidone/α-olefin copolymer is a polymer represented by the following formula: 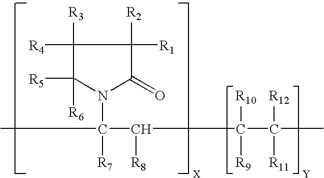
wherein the radicals R₁ to R₁₂ represent, independently one from another, a C₁₀ to C₄₀ linear or branched saturated alkyl group or a hydrogen atom, at least one of the aforementioned R₁ to R₁₂ being different from a hydrogen atom, X and Y are integers, and the value Y may be equal to 0 but X must not be equal to 0,
and further wherein the polyvinylpyrrolidone/α-olefin copolymer and the semi-crystalline polymer each are individually present in the composition in an amount of 0.1-14% by weight with respect to the total weight of the composition, the semi-crystalline polymer has a melting point of 50-120° C., and a number average molecular weight of 2,000 to 800,000, and the non-volatile oil is present in the composition in an amount of 1-99% by weight with respect to the total weight of the composition.

16. A method for producing a composition, comprising:
blending at least one semi-crystalline polymer having a melting point of at least 50° C., at least one polyvinylpyrrolidone/α-olefin copolymer having a melting point of at least 50° C., and an oil phase comprising at least one non-volatile oil;
gelifying said oil phase; and
applying a shear force after gelification and/or during gelification to obtain a fragmented gel structure.

17. A method, comprising applying the composition of claim 1 to human keratin material.

18. The method according to claim 17, wherein said keratin material is body or facial skin, the eyelashes and/or the lips.

19. A method, comprising applying the composition of claim 15 to human keratin material.

20. The method according to claim 19, wherein said keratin material is body or
facial skin, the eyelashes and/or the lips.
